(19) United States
(12) Plant Patent Application Publication
(10) Pub. No.: US 2019/0075698 P1
(43) Pub. Date: Mar. 7, 2019

(54) Hybrid tea rose plant named 'POULREN027'

(50) ;

(71) Applicant: Mogens Nyegaard Olesen, Fredensborg (DK)

(72) Inventor: Mogens Nyegaard Olesen, Fredensborg (DK)

(21) Appl. No.: 15/731,966

(22) Filed: Sep. 5, 2017

(51) Int. Cl. A01H 6/74

(52) U.S. Cl. PLT/140

(57) ABSTRACT

A new garden rose plant of the Hybrid Tea class which has abundant, red purple flowers and attractive foliage. This new and distinct variety has shown to be uniform and stable in the resulting generations from asexual propagation.

Botanical designation: Rosa hybrid.

Variety denomination: ‘Poulren027’.

### SUMMARY OF THE INVENTION

The present invention constitutes a new and distinct variety of rose plant which originated from a controlled crossing between the female seed parent, an unnamed seedling, and the male pollen parent, also an unnamed seedling. Both of the parent varieties are non-patented.

The two parents were crossed during the summer of 2007 and the resulting seeds were planted in a controlled environment in Fredensborg, Denmark. The new variety, named ‘Poulren027’, originated as a single seedling from the stated cross.

The new variety may be distinguished from its male pollen parent and female seed parent primarily by the following characteristics. The male pollen parent plant has red flowers while the new variety has red purple flowers. The female seed parent plant has light pink flowers while the new variety has red purple flowers.

The objective of the hybridization of this rose variety was to create a new and distinct variety with unique qualities, such as:
1. Uniform and abundant red purple flowers;
2. Vigorous, but compact growth when propagated on its own roots;
3. Exceptional disease resistance.

This combination of qualities is not present in previously available commercial cultivars of this type, known to the inventor, and distinguish ‘Poulren027’ from all other varieties of which we are aware.

As part of the rose development program, Mogens N. Olesen germinated the seeds from the aforementioned hybridization during winter of 2007 and conducted evaluations on the resulting seedlings in a controlled environment in Fredensborg, Denmark. ‘Poulren027’ was selected in the spring of 2008 by the inventor as a single plant from the progeny of the aforementioned hybridization.

Asexual reproduction of ‘Poulren027’ by rooted cuttings was first done by Mogens N. Olesen in the nursery in Fredensborg, Denmark in July, 2008. This initial and other subsequent asexual propagations conducted in controlled environments have demonstrated that the characteristics of ‘Poulren027’ are true to type and are transmitted from one generation to the next.

### DESCRIPTION OF THE DRAWING

The accompanying color illustrations show as true as is reasonably possible to obtain in color photographs of this type, the typical characteristics of the buds, flowers, leaves, and stems, of ‘Poulren027’.

Specifically illustrated in FIG. 1 of the drawings are open flowers, flower petals detached showing reproductive flower parts, sepals detached, and a flowering branch showing shape of flower buds.

FIG. 2 illustrates juvenile and mature stems and leaves. Plants shown are 2 years of age.

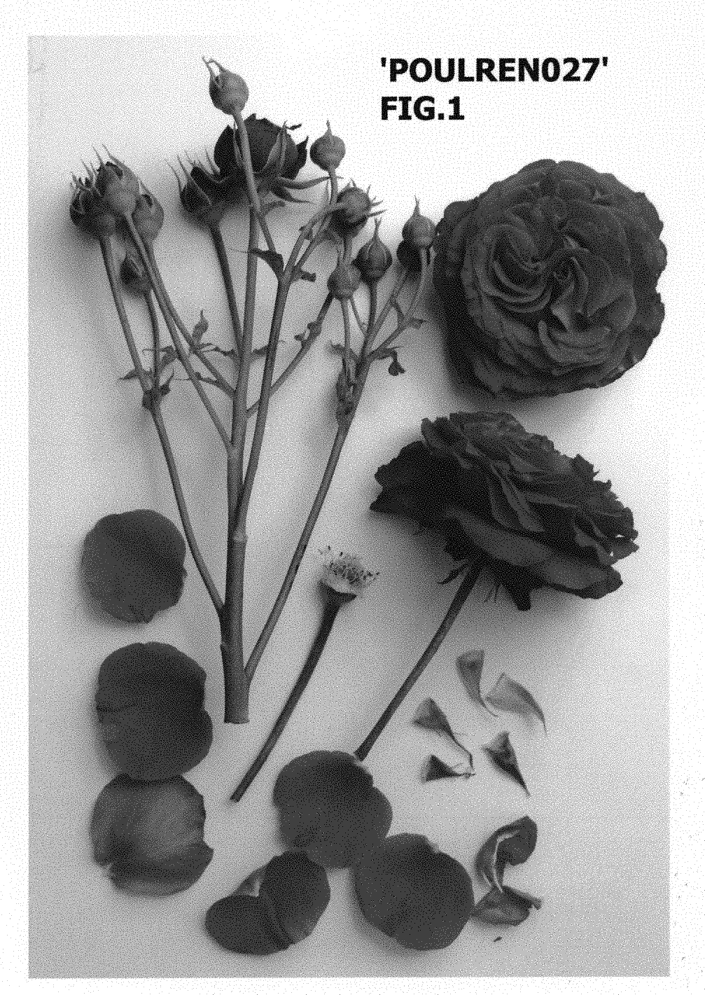

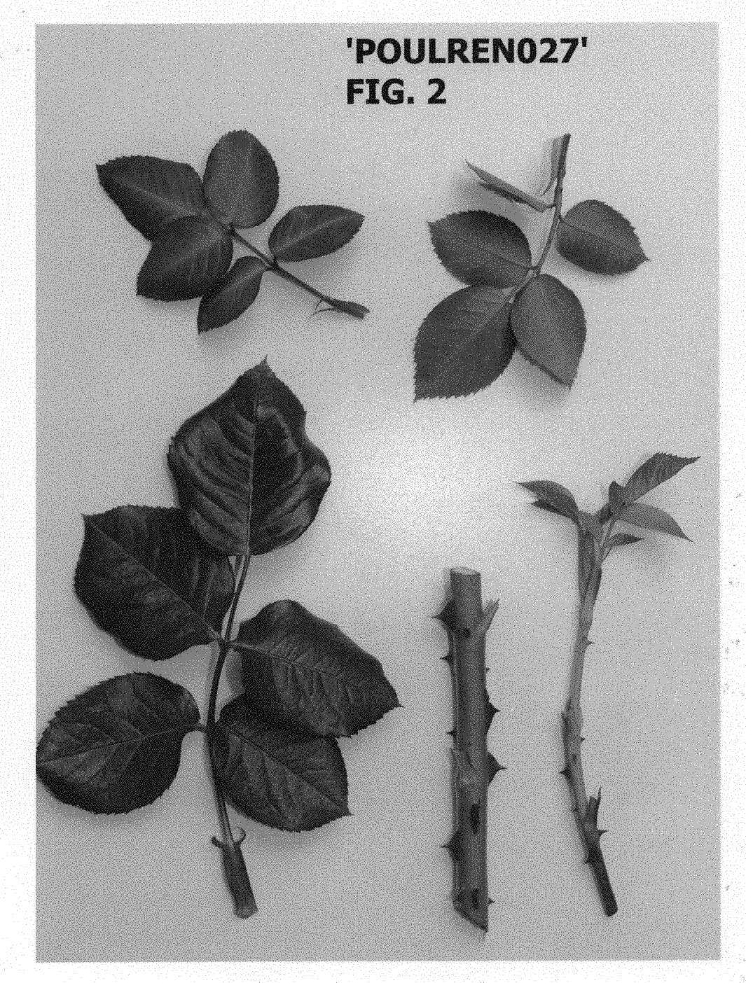

### DETAILED DESCRIPTION OF THE VARIETY

The following is a description of ‘Poulren027’, as observed in its growth in a field nursery in Marion County, Oreg. Observed plants are 2 years of age, and were grown on their own roots. Color references are made using The Royal Horticultural Society (London, England) Colour Chart, 2001, except where common terms of color are used.

For a comparison, several physical characteristics of the rose variety ‘Poulren021’, U.S. Plant Pat. No. 26,165 are compared to ‘Poulren027’ in Chart 1.

CHART 1

| | ‘Poulren027’ | ‘Poulren021’ |
|---|---|---|
| Petal Count | 55 | 60 |
| Flower Diameter | 115 mm | 100 mm |
| General Tonality | Red-Purple Group 60A | Red-Purple Group 61C |
| of Flower Color | | |

### FLOWER AND FLOWER BUD

Blooming habit: Continuous.
Flower bud:
   Size.—Upon opening, 33 mm in length from base of receptacle to end of bud. Bud diameter is 25 mm.
   Bud form.—Urceolate.
   Bud color.—As sepals divide petals are Greyed-Purple Group 183A and Red Group 53A.
   Sepal inner surface.—Color: Yellow-Green Group 144B with intonations of Greyed-Purple Group 184C. Surface: Lightly pubescent.
   Sepal outer surface.—Color: Yellow-Green Group 144A with intonations of Greyed-Purple Group 183B. Texture: Smooth.
   Sepal shape.—Apex: Cirrhose. Base: Flat at union with receptacle.
   Sepal margin.—Margins have weak foliaceous appendages on three of the five sepals.
   Sepal size.—35 mm long, 14 mm wide.
   Receptacle.—Texture: Smooth. Size: 7 mm in height, 15 mm wide. Color: Yellow-Green Group 144A with intonations of Greyed-Purple Group 184A. Shape: Broad campanulate.
   Pedicel.—Surface: Smooth. Length: About 75 mm. Diameter: 3 mm on average. Color: Yellow-Green Group 144B with intonations of Greyed-Purple Group 184A. Strength: Strong.
   Peduncle.—Length: 45 to 130 mm. Diameter: About 4 mm. Color: Yellow-Green Group 144A with intonations of Greyed-Red Group 180A. Texture: Smooth.
Flower bud development: Flower buds are borne in clusters of about 15 flower buds per stem. Flower bloom:
   Fragrance.—Strong perfume.
   Duration.—The blooms have a duration on the plant of approximately 10 days. Petals fall cleanly away from plant after flowers have fully matured.
   Size.—Flower diameter is 115 mm when open. Flower depth is 60 mm.
   Flower shape.—Quartered-rosette, very double, with many overlapping petals packed into quarter sections.
   Shape of flower, side view.—The upper portion is convex. The lower portion is a flat convex.
Petalage: Under normal conditions, flowers have about 55 petals.
General tonality of flower: Open flowers are Red-Purple Group 60A.
Petal color:
   Upon opening, outer petals.—Upper surface: Red-Purple Group 61B. Lower surface: Red-Purple Group 64A.
   Upon opening, inner petals.—Upper surface: Red-Purple Group 61B. Lower surface: Red-Purple Group 64A.
   Basal petal spots, upon opening.—Upper surface: Yellow Group 4D. Lower surface: Yellow Group 4D.
   After opening, outer petals.—Upper surface: Red-Purple Group N57B. Lower surface: Red-Purple Group 64C.
   After opening, inner petals.—Upper surface: Red-Purple Group N57B. Lower surface: Red-Purple Group 64C.
   Basal petal spots, after opening.—Upper surface: Yellow Group 4D. Lower surface: Yellow Group 4D.
Petals:
   Petal reflex.—Somewhat reflexed.
   Margin.—Entire and uniform. Moderate undulations.
   Shape.—Broad and rounded. Apex shape: Rounded. Base shape: Rounded.
   Size.—60 mm (l) x 65 mm (w).
   Texture.—Smooth.
   Thickness.—Average.
Petaloids:
   Size.—45 mm (l) by 25 mm (w).
   Quantity.—About 10.
   Shape.—Elliptical with an acute base and rounded apex.
   Color.—Upper surface Red-Purple Group N57B. Lower surface Red-Purple Group 64C.
Reproductive flower parts:
   Pollen.—None observed.
   Anthers.—Size: 3 mm in length. Color: Greyed-Orange Group 163B. Quantity: 40 on average.
   Filaments.—Color: White Group 155C with intonations of Greyed-Red Group 181C. Length: 8mm.
   Pistils.—Length: 13 mm. Quantity: 45 on average.
   Stigmas.—Color: Orange-White Group 159B.
   Styles.—Color: Red-Purple Group 59D.
   Location of stigmas.—Superior in location relative to the length of the filaments and the height of the anthers.
   Hips.—None Observed.

### PLANT

Plant growth: Upright, bushy. Plants are 130 cm in height, and 100 cm wide.
Stems:
   Color of juvenile growth.—Yellow-Green Group 144B.
   Color of mature growth.—Yellow-Green Group 144A.
   Length.—Canes are about 65 cm from the base of the plant to the flowering portion.
   Diameter.—About 12 mm.
   Internodes.—On mature canes about 50 to 60 mm between nodes.
   Surface texture.—Young wood: Smooth. Older wood: Smooth.
Long prickles:
   Incidence.—11 prickles per 10 cm of stem.
   Size.—Average length of prickles on mature stems is 6 mm by 10 mm wide.
   Shape.—Upper portion is linear. Lower portion is concave.
   Color.—Juvenile prickles: Greyed-Red Group 182A. Mature prickles: Greyed-Red Group 182A.
Plant foliage:
   Compound leaf.—190 mm (l) x 125 (w).
   Quantity.—2 leaves per 10 cm of stem on average.
   Leaf bearing angle to the stem.—45 degrees.
   Color of juvenile foliage: Upper side: Yellow-Green Group 153D with intonations of Greyed-Orange Group 175B. Lower side: Yellow-Green Group 153D with intonations of Greyed-Red Group 181A.
   Color of mature foliage.—Upper side: Yellow-Green Group 147A. Lower side: Yellow-Green Group 147B.
Plant leaves and leaflets:
   Stipules.—Size: 32 mm long, 5 mm wide. Quantity: 2 per compound leaf. Shape: Linear, slightly broad based with outward extending apices. Margins: Finely serrated. Color: Greyed-Orange Group 166A.
   Petiole.—Length: 60 mm. Diameter: 2 mm. Upper surface color: Yellow-Green Group 144A with intonations of Greyed-Red Group 181A. Lower surface color: Yellow-Green Group 144A.
   Rachis.—Length: 65 mm. Upper surface color: Yellow-Green Group 144A with intonations of Greyed-Red Group 181A. Lower surface color: Yellow-Green Group 144A.
   Leaflet.—Quantity: Normally 5 leaflets. Margins: Serrated. Size: Terminal leaflets are about 70 mm long, 55 mm wide. Shape: Generally elliptical. Base: Rounded. Apex: Acute. Texture: Smooth. Thickness: Average. Arrangement: Odd pinnate. Venation: Reticulate. Glossiness: Very glossy.
Disease resistance: Above average resistance to powdery mildew Sphaerotheca pannosa, downy mildew Peronospora sparsa, rust Phragmidium sps., black spot Diplocarpon rosae, and Botrytis cinerea under normal growing conditions.
Cold hardiness: The variety is tolerant to USDA Cold Hardiness Zone 6.
Heat tolerance: The variety has been found to be suitable for climate conditions found in the American Horticulture Society heat zone 7.

We claim:

1. A new and distinct variety of rose plant of the Hybrid Tea rose class named ‘Poulren027’, substantially as illustrated and described herein, due to its abundant red purple flowers, disease resistance, and extended period of bloom.
